(19) United States
(12) Patent Application Publication
(10) Pub. No.: US 2018/0296095 A1
(43) Pub. Date: Oct. 18, 2018

(54) CHARACTERIZING LIFE QUALITY OF A LIVING ENTITY VIA HYPER-SPECTRAL IMAGING AND ANALYSIS, AND APPLICATIONS THEREOF

(71) Applicant: Green Vision Systems Ltd., Tel-Aviv (IL)

(72) Inventor: Danny S. MOSHE, Tel-Aviv, IL (US)

(21) Appl. No.: 14/788,950

(22) Filed: Jul. 1, 2015

Related U.S. Application Data
(60) Provisional application No. 62/019,462, filed on Jul. 1, 2014.

(51) Int. Cl. A61B 5/00

(52) U.S. Cl. CPC A61B 5/0075; A61B 5/7246; A61B 5/486; A61B 2562/028; A61B 2503/40; A61B 2576/00; A61B 2560/0475; A61B 5/6898

(57) ABSTRACT

Methods and apparatuses for characterizing life quality of a living entity via hyper-spectral imaging and analysis, and applications thereof, such as managing life quality of a living entity. Includes acquiring hyper-spectral imaging data and information of: anatomical features of the living entity, and substances consumable by the living entity; generating and maintaining a living entity-specific database containing data and information about the living entity; processing acquired living entity anatomical feature and acquired living entity consumable substance hyper-spectral imaging data and information, and living entity data and information; using processed data and information to generate living entity life quality data and information characteristic of life quality of the living entity. Applicable to any live or living entity (human, animal, plant). Applicable as a stand-alone system or as integrated microelectromechanical (MEM) [chip level] components as part of a smart or intelligent device.

### RELATED APPLICATION

This application claims the benefit of priority under 35 USC § 119(e) of U.S. Provisional Patent Application No. 62/019,462 filed on Jul. 1, 2014, the contents of which are incorporated herein by reference in their entirety.

### FIELD OF THE INVENTION

The present invention, in some embodiments thereof, relates to life quality characterization of a living entity, and applications thereof, and more particularly, but not exclusively, to methods and apparatuses for characterizing life quality of a living entity via hyper-spectral imaging and analysis, and applications thereof, such as managing life quality of a living entity. Accordingly, the present invention, in some embodiments thereof, is directed to subject matter corresponding to a unique intersection of the fields and arts relating to: (i) characterizing life quality of a living entity, (ii) managing life quality of a living entity, and (iii) hyper-spectral imaging and analysis. Exemplary embodiments of the present invention are applicable for being practiced whereby the living entity is any live or living entity, such as a live human being, a live animal, or a live plant.

### BACKGROUND OF THE INVENTION

Life quality of a living entity (human, animal, or plant) is associated with/related to a wide variety of numerous different categories of subjects, activities, and data-information about the living entity. Among them are two important categories of: (1) anatomical features of the living entity, and (2) substances consumable by the living entity.

Regarding anatomical features of the living entity, examples are those which are typically exposed and readily viewable to an observer. For example, in the case of the living entity being a live human or live animal, exemplary anatomical features are (partly or fully) exposed skin, hair, or/and nails. For example, in the case of the living entity being a live plant, exemplary anatomical features are (partly or fully) exposed stems, stalks, branches, leaves, or/and flowers.

Regarding substances consumable by the living entity, examples are those substances, materials, or objects which are typically required and consumed by the living entity for maintaining basic good health and life thereof, such as food, water, and air.

It is well established and known that characteristics and properties of such exemplary anatomical features of a living entity are (directly or indirectly) affected by characteristics and properties of such exemplary substances, materials, or objects consumable by the living entity.

Accordingly, aspects of life quality of a living entity are (directly or indirectly) affected by characteristics and properties of such exemplary anatomical features of the living entity and of such exemplary substances, materials, or objects consumable by the living entity.

Of wide and general potential interest, and application, is for one to have the ability or capability to characterize aspects of life quality of a living entity, where such ability or capability can be used for (directly or indirectly) assisting in managing life quality of the living entity, with an ultimate objective or goal of improving life quality of the living entity.

### Hyper-Spectral Imaging and Analysis

Hyper-spectral imaging and analysis has been established as a highly unique, specialized, and sophisticated, combined spectroscopy and imaging type of analytical method or technique, in the more encompassing field or area of analytical science and technology, involving the sciences and technologies of spectroscopy and imaging. By definition, hyper-spectral imaging and analysis is based on a combination of spectroscopy and imaging theories, principles, and practices, which are exploitable for analyzing and classifying various different types and kinds of samples of matter in a highly unique, specialized, and sophisticated, manner.

Hyper-spectral imaging, in general, generating and collecting hyper-spectral images, and, processing and analyzing hyper-spectral image data and information, in particular, theory, principles, and practices thereof, and, related and associated applications and subjects thereof, such as the more general subject of spectral imaging, are well known and taught about in scientific, technical, and patent, literature, and currently practiced in a wide variety of numerous different fields and areas of technology. Exemplary teachings and practices of hyper-spectral imaging and analysis by the same applicant/assignee of the present disclosure are provided in references 1-9 (and references cited therein).

In sharp contrast to the regular or standard spectroscopic imaging technique of ‘spectral’ imaging and analysis, the more highly specialized, complex, and sophisticated, spectroscopic imaging technique of ‘hyper-spectral’ imaging and analysis, consists of using a hyper-spectral imaging and analysis system for on-line (real time, near-real time) or off-line generating and collecting (acquiring) hyper-spectral images and spectra (herein, together, generally referred to as hyper-spectral imaging data and information), and, processing and analyzing the acquired hyper-spectral imaging data and information.

In hyper-spectral imaging, multiple fields of view of an object (and components thereof) are ‘hyper-spectrally’ scanned and imaged while the object (and components thereof) is exposed to electromagnetic radiation.

During the hyper-spectral scanning and imaging there is generating and collecting relatively large numbers (up to the order of millions) of multiple spectral (i.e., hyper-spectral) images, ‘one-at-a-time’, but, in an extremely fast or rapid sequential manner, of the object (and components thereof) emitting electromagnetic radiation at a plurality of many wavelengths (or frequencies, or energies), where the wavelengths (or frequencies, or energies) are associated with different selected (relatively narrow) portions or bands, or bands therein, of an entire hyper-spectrum emitted by the object (and components thereof). A hyper-spectral imaging and analysis system can be operated in an extremely fast or rapid manner for providing exceptionally highly resolved spectral and spatial data and information of an imaged object (and components thereof), with high accuracy and high precision (reproducibility), which are fundamentally unattainable by using a regular or standard spectral imaging and analysis system.

In general, when electromagnetic radiation, for example, in the form of light such as that supplied by the sun, or by a man-made imaging type of illuminating or energy source, such as that used during hyper-spectral imaging, is incident upon an object, the electromagnetic radiation is affected by one or more of the components making up the object, by any combination of electromagnetic radiation absorption, diffusion, reflection, diffraction, scattering, or/and transmission, mechanisms. Moreover, an object whose composition includes organic chemical species or components, ordinarily exhibits some degree or extent of fluorescent or/and phosphorescent properties, characteristics, and behavior, when illuminated by some type of electromagnetic radiation or light, such as ultra-violet (UV), visible (VIS), or infrared (IR), types of light. The affected electromagnetic radiation, in the form of diffused, reflected, diffracted, scattered, or/and transmitted, electromagnetic radiation emitted by, or/and emerging from, the object (and components thereof), is directly and uniquely related to, and can be correlated with, the physical, chemical, or/and biological properties, characteristics, and behavior, of the object, in general, and of the components making up the object, in particular, and therefore represents a spectral ('fingerprint' or ‘signature’) pattern type of identification and characterization of the object, which is directly applicable for analyzing and classifying the object.

Accordingly, hyper-spectral images generated by, and collected from, an object (and components thereof) are correlated with emission spectra of the object (and components thereof), where the emission spectra correspond to spectral representations in the form of spectral ‘fingerprint’ or ‘signature’ pattern types of identification and characterization, of the hyper-spectrally imaged object (and components thereof). Such hyper-spectral image data and information are processed and analyzed by using automatic pattern recognition (APR) or/and optical character recognition (OCR) types of hyper-spectral imaging data and information processing and analysis, for identifying, characterizing, or/and classifying, the physical, chemical, or/and biological properties, characteristics, and behavior, of the hyper-spectrally imaged object (or/and components thereof).

### SUMMARY OF THE INVENTION

The present invention, in some embodiments thereof, relates to methods and apparatuses for characterizing life quality of a living entity via hyper-spectral imaging and analysis, and applications thereof, such as managing life quality of a living entity. Exemplary embodiments of the present invention are applicable for being practiced whereby the living entity is any live or living entity, such as a live human being, a live animal, or a live plant. Exemplary embodiments of the present invention are applicable for being practiced whereby the living entity is any live or living entity, such as a live human being, a live animal, or a live plant. Exemplary embodiments of the present invention are applicable for being implemented in the form of a single, stand-alone multi-assembly or multi-device type of system, or, alternatively, in the form of (integrated) microelectromechanical (MEM) [chip level] components as part of any one of various different types or kinds of a smart or intelligent device, such as a smart or intelligent phone, a smart or intelligent wallet, a smart or intelligent watch, a smart or intelligent hand bracelet, or a smart or intelligent finger ring, among other possible smart or intelligent devices, used by people. Exemplary embodiments of the present invention are applicable for being implemented via local based communication methods, protocols, and equipment, or/and via web/internet/cloud based communication methods, protocols, and equipment.

According to an aspect of some embodiments of the present invention there is provided a method for characterizing life quality of a living entity via hyper-spectral imaging and analysis, the method comprising: acquiring hyper-spectral imaging data and information of: at least one anatomical feature of the living entity, and at least one substance consumable by the living entity; generating and maintaining a living entity-specific database containing data and information about the living entity; processing the acquired living entity anatomical feature hyper-spectral imaging data and information, the acquired living entity consumable substance hyper-spectral imaging data and information, and the data and information about the living entity; and using the processed data and information to generate living entity life quality data and information characteristic of at least one aspect of life quality of the living entity.

According to some embodiments of the invention, the acquiring, the generating and maintaining, the processing, and the using, are performed according to a chronological sequence or plurality of a number of times, spanning a pre-determined period or length of time.

According to some embodiments of the invention, the pre-determined period or length of time is on order of minutes, hours, days, weeks, months, or years.

According to some embodiments of the invention, the acquiring, the generating and maintaining, the processing, and the using, are performed according to a real-time manner or mode of operation.

According to some embodiments of the invention, the acquiring, the generating and maintaining, the processing, and the using, are performed as part of a single, multi-assembly or multi-device system.

According to some embodiments of the invention, the acquiring, the generating and maintaining, the processing, and the using, are performed in a form of integrated microelectromechanical (MEM) [chip level] components as part of a smart or intelligent device.

According to some embodiments of the invention, the smart or intelligent device is selected from the group consisting of smart or intelligent phones, smart or intelligent wallets, smart or intelligent watches, smart or intelligent hand bracelets, and smart or intelligent finger rings.

According to some embodiments of the invention, the processing includes forming sets and databases of interferogram images from the acquired living entity anatomical feature hyper-spectral imaging data and information, the acquired living entity consumable object hyper-spectral imaging data and information, and the data and information about the living entity.

According to some embodiments of the invention, the processing includes segmenting, via analyzing and classifying, as a function of time: (i) elements of the acquired living entity anatomical feature hyper-spectral imaging data and information, (ii) elements of the acquired living entity consumable object hyper-spectral imaging data and information, and (iii) elements of the living entity-specific database containing reference data and information about the living entity.

According to some embodiments of the invention, the using includes correlating at least two of: (i) the segmented acquired living entity anatomical feature hyper-spectral imaging data and information, (ii) the segmented acquired living entity consumable object hyper-spectral imaging data and information, and (iii) the segmented reference data and information about the living entity.

According to some embodiments of the invention, following the correlating, at least some elements of the generated living entity life quality data and information characteristic of at least one aspect of life quality of the living entity are fed back to the processing, so as to update or/and modify the processing of the acquired living entity anatomical feature hyper-spectral imaging data and information, the acquired living entity consumable object hyper-spectral imaging data and information, and the data and information about the living entity.

According to some embodiments of the invention, following the correlating, at least some elements of the processed data and information of the acquired living entity anatomical feature hyper-spectral imaging data and information, the acquired living entity consumable object hyper-spectral imaging data and information, and the data and information about the living entity, are fed back to the generating and maintaining, so as to update or/and modify the generating and maintaining the living entity-specific database containing data and information about the living entity.

According to some embodiments of the invention, the living entity life quality data and information characteristic of at least one aspect of life quality of the living entity is used for assisting in managing life quality of the living entity.

According to another aspect of some embodiments of the present invention there is provided an apparatus for characterizing life quality of a living entity via hyper-spectral imaging and analysis, the apparatus comprising: a hyper-spectral imaging unit, configured to acquire hyper-spectral imaging data and information of: at least one anatomical feature of the living entity, and at least one substance consumable by the living entity; a living entity-specific database containing data and information about the living entity; a data-information processing unit, configured to: process the acquired living entity anatomical feature hyper-spectral imaging data and information, the acquired living entity consumable substance hyper-spectral imaging data and information, and the data and information about the living entity; and generate living entity life quality data and information characteristic of at least one aspect of life quality of the living entity.

According to some embodiments of the invention, the hyper-spectral imaging unit, and the data-information processing unit are configured to operate according to a chronological sequence or plurality of a number of times, spanning a pre-determined period or length of time.

According to some embodiments of the invention, the pre-determined period or length of time is on order of minutes, hours, days, weeks, months, or years.

According to some embodiments of the invention, the hyper-spectral imaging unit, the living entity-specific database, and the data-information processing unit are configured to operate according to a real-time manner or mode of operation.

According to some embodiments of the invention, the hyper-spectral imaging unit, the living entity-specific database, and the data-information processing unit are configured as part of a single, multi-assembly or multi-device system.

According to some embodiments of the invention, the hyper-spectral imaging unit, the living entity-specific database, and the data-information processing unit are configured in a form of integrated microelectromechanical (MEM) [chip level] components as part of a smart or intelligent device.

According to some embodiments of the invention, the smart or intelligent device is selected from the group consisting of smart or intelligent phones, smart or intelligent wallets, smart or intelligent watches, smart or intelligent hand bracelets, and smart or intelligent finger rings.

According to some embodiments of the invention, the data-information processing unit is configured to process the data and information by forming sets and databases of interferogram images from the acquired living entity anatomical feature hyper-spectral imaging data and information, the acquired living entity consumable object hyper-spectral imaging data and information, and the data and information about the living entity.

According to some embodiments of the invention, the data-information processing unit is configured to process the data and information by segmenting, as a function of time: (i) elements of the acquired living entity anatomical feature hyper-spectral imaging data and information, (ii) elements of the acquired living entity consumable object hyper-spectral imaging data and information, and (iii) elements of the living entity-specific database containing reference data and information about the living entity.

According to some embodiments of the invention, the data-information processing unit is further configured to correlate at least two of: (i) the segmented acquired living entity anatomical feature hyper-spectral imaging data and information, (ii) the segmented acquired living entity consumable object hyper-spectral imaging data and information, and (iii) the segmented reference data and information about the living entity.

According to some embodiments of the invention, the data-information processing unit is configured to update or/and modify at least some elements of the processed data and information of the acquired living entity anatomical feature hyper-spectral imaging data and information, the acquired living entity consumable object hyper-spectral imaging data and information, and the data and information about the living entity.

According to some embodiments of the invention, the apparatus further comprises a controller-processor configured to control the hyper-spectral imaging unit.

According to some embodiments of the invention, the controller-processor is operatively connected to the hyper-spectral imaging unit and to the data-information processing unit.

According to some embodiments of the invention, the data and information about the living entity is transferred from the living entity-specific database to the data-information processing unit.

According to some embodiments of the invention, the living entity life quality data and information is fed back to the data-information processing unit.

According to some embodiments of the invention, the data-information processing unit is configured to feed back the processed data and information to the living entity-specific database.

According to some embodiments of the invention, data and information, or/and control signals, are exchanged between the data-information processing unit and the hyper-spectral imaging unit.

According to some embodiments of the invention, data and information, or/and control signals, are exchanged between the controller-processor and the hyper-spectral imaging unit.

According to some embodiments of the invention, data and information, or/and control signals, are exchanged between the data-information processing unit and the controller-processor.

According to some embodiments of the invention, the data-information processing unit is configured to enable using the living entity life quality data and information to assist in managing life quality of the living entity.

According to another aspect of some embodiments of the present invention there is provided a method of assisting in managing life quality of a living entity via hyper-spectral imaging and analysis, the method comprising: characterizing life quality of the living entity via a method of hyper-spectral imaging and analysis, so as to generate living entity life quality data and information characteristic of at least one aspect of life quality of the living entity; and using the living entity life quality data and information to assist in the managing of life quality of the living entity.

According to some embodiments of the invention, the characterizing life quality of the living entity is performed according to a method comprising: acquiring hyper-spectral imaging data and information of: at least one anatomical feature of the living entity, and at least one substance consumable by the living entity; generating and maintaining a living entity-specific database containing data and information about the living entity; processing the acquired living entity anatomical feature hyper-spectral imaging data and information, the acquired living entity consumable substance hyper-spectral imaging data and information, and the data and information about the living entity; and using the processed data and information to generate living entity life quality data and information characteristic of at least one aspect of life quality of the living entity.

According to another aspect of some embodiments of the present invention there is provided an apparatus for assisting in managing life quality of a living entity via hyper-spectral imaging and analysis, the apparatus comprising: an apparatus for characterizing life quality of the living entity via hyper-spectral imaging and analysis, configured to generate living entity life quality data and information characteristic of at least one aspect of life quality of the living entity; and a data-information processing unit, configured to enable using the living entity life quality data and information to assist in the managing of life quality of the living entity.

According to some embodiments of the invention, the apparatus comprises: a hyper-spectral imaging unit, configured to acquire hyper-spectral imaging data and information of: at least one anatomical feature of the living entity, and at least one substance consumable by the living entity; a living entity-specific database containing data and information about the living entity; a data-information processing unit, configured to: process the acquired living entity anatomical feature hyper-spectral imaging data and information, the acquired living entity consumable substance hyper-spectral imaging data and information, and the data and information about the living entity; and generate living entity life quality data and information characteristic of at least one aspect of life quality of the living entity.

All technical and/or scientific words, terms, or/and phrases, used herein throughout the present disclosure have the same or similar meaning as commonly understood by one of ordinary skill in the art to which the invention pertains, unless otherwise specifically defined or stated herein. Although materials or/and methods equivalent or similar to those described herein can be used in practicing or/and testing embodiments of the invention, exemplary materials or/and methods are described below.

In case of conflict, the patent specification, including definitions, will control. In addition, materials, methods, and examples described herein are illustrative only and are not intended to be necessarily limiting.

As will be appreciated by one skilled in the art, some embodiments of the present invention may be embodied as a system, method or computer program product.

Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system”.

Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof.

Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, such as signal processing and matching, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWINGS

Some embodiments of the present invention are herein described, by way of example only, with reference to the accompanying drawings. With specific reference now to the drawings in detail, it is stressed that the particulars shown are by way of example and for purposes of illustrative description of some embodiments of the present invention. In this regard, the description taken together with the accompanying drawings make apparent to those skilled in the art how some embodiments of the present invention may be practiced.

In the drawings:

FIG. 1 is a flow diagram of a method for characterizing life quality of a living entity via hyper-spectral imaging and analysis, and applications thereof, in accordance with some embodiments of the invention;

FIG. 2 is a (block-type) schematic diagram of an apparatus for characterizing life quality of a living entity via hyper-spectral imaging and analysis, and applications thereof, in accordance with some embodiments of the invention;

FIG. 3 is a (block-type) schematic diagram of an apparatus for characterizing life quality of a living entity via hyper-spectral imaging and analysis, and applications thereof, highlighting an exemplary ‘chip level’ type design and construction, configurable and operable with integrated microelectromechanical (MEM) [chip level] components as part of an exemplary smart device, in accordance with some embodiments of the invention; and

FIG. 4 is a schematic diagram illustrating an apparatus for characterizing life quality of a living entity via hyper-spectral imaging and analysis, and applications thereof, as an example of the apparatus of FIG. 3, configured into an exemplary smart phone, in accordance with some embodiments of the invention.

### DESCRIPTION OF SPECIFIC EMBODIMENTS OF THE INVENTION

The present invention, in some embodiments thereof, relates to life quality characterization of a living entity, and applications thereof, and more particularly, but not exclusively, to methods and apparatuses for characterizing life quality of a living entity via hyper-spectral imaging and analysis, and applications thereof, such as managing life quality of a living entity. Accordingly, the present invention, in some embodiments thereof, is directed to subject matter corresponding to a unique intersection of the fields and arts relating to: (i) characterizing life quality of a living entity, (ii) managing life quality of a living entity, and (iii) hyper-spectral imaging and analysis.

Exemplary embodiments of the present invention are applicable for being practiced whereby the living entity is any live or living entity, such as a live human being, a live animal, or a live plant. Exemplary embodiments of the present invention are applicable for being implemented in the form of a single, stand-alone multi-assembly or multi-device type of system, or, alternatively, in the form of (integrated) microelectromechanical (MEM) [chip level] components as part of any one of various different types or kinds of a smart or intelligent device, such as a smart or intelligent phone, a smart or intelligent wallet, a smart or intelligent watch, a smart or intelligent hand bracelet, or a smart or intelligent finger ring, among other possible smart or intelligent devices, used by people.

Exemplary embodiments of the present invention are applicable for being implemented via local based communication methods, protocols, and equipment, or/and via web/internet/cloud based communication methods, protocols, and equipment.

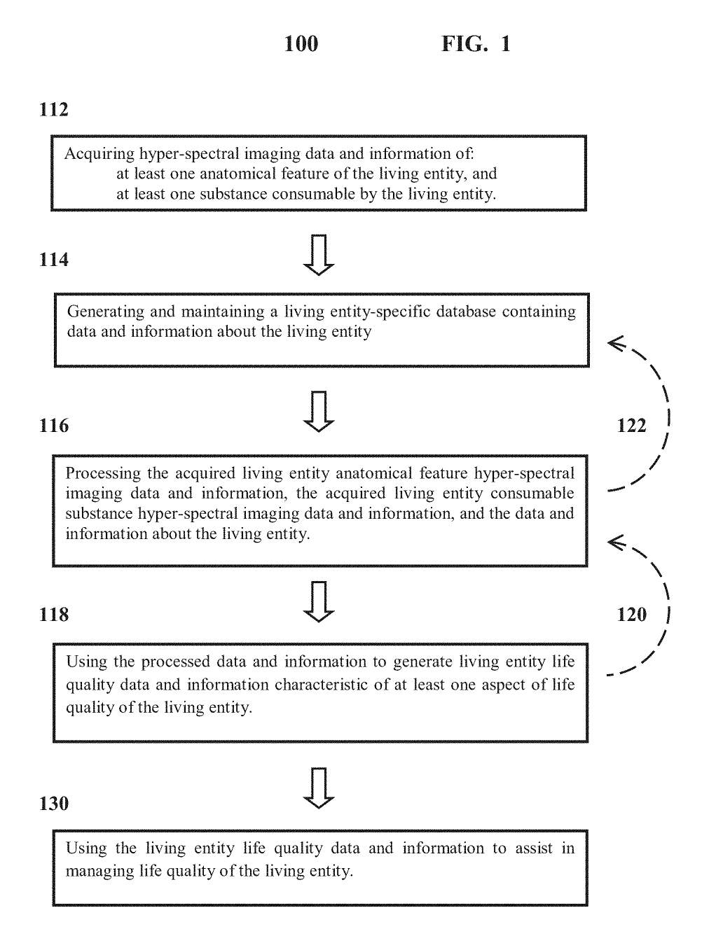

In the following illustrative description of some embodiments of the invention, reference is made to the figures (FIGS. 1-4). Throughout the following description and accompanying drawings, same reference numbers refer to same components, elements, or features. It is to be understood that the invention is not necessarily limited in its application to the details of construction and the arrangement of the components and/or methods set forth in the following illustrative description. The invention is capable of other embodiments or of being practiced or carried out in various ways.

Additionally, it is to be fully understood that certain aspects, characteristics, and features, of the present invention, which are illustratively described and presented in the context or format of a plurality of separate embodiments, may also be illustratively described and presented in any suitable combination or sub-combination in the context or format of a single embodiment. Conversely, various aspects, characteristics, and features, of the present invention, which are illustratively described and presented in combination or sub-combination in the context or format of a single embodiment, may also be illustratively described and presented in the context or format of a plurality of separate embodiments.

For example, the following includes illustrative description of several aspects of some embodiments of the invention. Specifically, the following presentation includes illustrative description of some embodiments of a method for characterizing life quality of a living entity via hyper-spectral imaging and analysis, and some embodiments of an apparatus (in the form of a system or of a small device) for characterizing life quality of a living entity via hyper-spectral imaging and analysis. It is to be understood that some embodiments of the disclosed method may be implemented independent of the disclosed apparatus, or by using some embodiments of the disclosed apparatus, and vice versa.

Accordingly, it is to be understood that illustrative description of one or more particular aspects of some embodiments of the invention may be considered independent or dependent of the respective illustrative description of one or more particular aspects of some other embodiments of the invention.

### Living (Live) Entities

The phrase ‘living entity’, as used herein, refers to any living (live) entity, such as a living (live) human being, a living (live) animal, or, a living (live) plant. The phrase ‘living entity’, as used herein, is synonymous with, and equivalent to, the phrase ‘live entity’.

The living entity can be of any age, from a newly (new) born or formed living entity, to an old or aged living entity.

For example, the living entity may be a live human being, for example, selected from the group consisting of a newly (new) born baby (infant), a baby (infant), a child, an adolescent, a young adult, an adult, a middle aged person, and an old-aged (elderly) person.

For example, the living entity may be a live animal member of the animal kingdom (phylum, class, order, family, genus, or species thereof), of any age.

For example, the living entity may be a live plant member of the plant kingdom (phylum/division, class, order, family, genus, or species thereof), of any age.

### Anatomical Features/Body Parts

The phrase ‘anatomical feature’, as used herein, refers to essentially any anatomical feature, or part of the body, of a living entity which is or can be (partly or fully) exposed and at least partly viewable by an observer, either with or without movement or manipulation of the body of the living entity or/and removal of something covering the body of the living entity. The phrase ‘anatomical feature’, as used herein, is synonymous with, and equivalent to, the phrase ‘part of the body’, unless specifically stated otherwise.

In the case of the living entity being a living human or living animal, exemplary anatomical features are those parts of the human or animal body which are typically (partly or fully) exposed and at least partly viewable by an observer, such as skin, hair, nails, teeth.

Additional exemplary anatomical features of a living human or living animal are those body parts associated with an eye, such as an eyelid and an eyelash.

In the case of the living entity being a living plant, exemplary anatomical features are those parts of the plant body which are typically (partly or entirely) exposed and at least partly viewable by an observer, such as stems, stalks, branches, leaves, flowers, or/and roots.

### Consumable Substances, Materials, Objects

The phrase ‘substance consumable by the living entity’, or, alternatively, the phrase ‘consumable substance’, as used herein, refers to essentially any substance, material, or object, which is consumable or usable (i.e., may be consumed or used) by a living entity in a manner where the consumable substance, material, or object, contacts (touches, comes into direct contact with, or is ingested by) at least part of the body of the living entity.

The phrases ‘substance consumable’ and ‘consumable substance’ are herein used in a synonymous and equivalent manner, and it is to be understood that appearance and meaning of either of these phrases are equivalent to appearance and meaning of the other phrase.

Moreover, the phrases ‘substance consumable’ and ‘consumable substance’, as used herein, are synonymous with, and equivalent to, the phrases ‘material consumable’, ‘consumable material’, ‘object consumable’, and ‘consumable object’, unless specifically stated otherwise.

Accordingly, it is to be understood that appearance and meaning of any of these phrases are equivalent to appearance and meaning of the other phrases, unless specifically stated otherwise.

Exemplary consumable substances, materials, or objects, are those which are typically required and consumed by a living entity for regularly (daily) maintaining basic good health, well being, and quality of life thereof.

Additional exemplary consumable objects, materials, or substances, are those which are consumed by a living entity for also maintaining, or/and improving, or/and enhancing, or/and treating, health, well being, and quality of life thereof.

Such exemplary consumable substances, materials, or objects, are selected from the group consisting of food (and food products), water (and water products), air, clothing, bedding, toiletries (bathroom products), cosmetics (cosmetic products), and pharmaceuticals (pharmaceutical products)/medicines (medicine [medicinal] products).

### Foods/Food Products

Exemplary (consumable) foods (and food products) are essentially any food item or product which is obtained and consumable (edible) in its natural state, or consumable (edible) after processing (cleaning, spicing, cooking, baking, etc.). Exemplary (consumable) foods (and food products) are essentially any food item or product which is derived from one or more naturally existing agricultural products. Exemplary (consumable) foods (and food products) are essentially any food item or product which is partly or wholly synthetically made or manufactured.

Examples of the above stated types or kinds of exemplary (consumable) foods (and food products) are selected from the group consisting of grains (and grain products), fruits (and fruit products), vegetables (and vegetable products), fish (and fish products), meat (and meat products], and dairy products.

Any of the above listed exemplary (consumable) foods (and food products) may be in essentially any physicochemical form of a solid, or a liquid, or a combination thereof.

### Water/Water Products

Exemplary (consumable) water (and water products) are essentially any water-based liquid which is consumable (edible). Exemplary (consumable) water (and water products) are tap (faucet) water, filtered water, water-based beverages, and water-based drinks.

### Air

Exemplary (consumable) air is outdoor air, or indoor air present inside of a residential building (dwelling), such as a home or apartment, or air inside of a commercial (office) building, and which comes into contact with a living entity. Exemplary (consumable) air is air that is processed, re-processed, circulated, or/and generated, by an air filtration device, or a conditioner. Such air typically contains dust and other air particular matter, among other substances, materials, and objects, possibly present in the (consumable) air.

### Clothing/Garments

Exemplary (consumable) clothing (garments) are essentially any type or kind of outer wear clothing (garments), or under wear clothing (garments).

### Bedding/Bedclothes

Exemplary (consumable) bedding (bedclothes) are sheets, blankets, and other coverings of a bed, such as pillow cases, that are ordinarily used with a bed for a living entity to sleep.

### Toiletries/Bathroom Products

Exemplary (consumable) toiletries/bathroom products are soaps, soap products, and related soap type body cleaning products, shampoos, hair conditioners, and related hair cleaning/conditioning type products, toothpaste, mouth wash and related mouth cleaning type products, toothbrushes and related mouth cleaning type products, hair combs, hair brushes, body towels, hand towels, toilet paper, wash cloths, tissues, deodorants, and antiperspirants.

### Cosmetics/Cosmetic Products

Exemplary (consumable) cosmetics (cosmetic products) are facial makeup, and body/skin/facial/leg/hand powders, ointments, creams, lotions, gels, oils. Additional exemplary (consumable) cosmetics (cosmetic products) are formulations (sunscreens, sun blockers, and the like) applied to prevent or minimize UV radiation from damaging the skin.

### Pharmaceuticals/Pharmaceutical Products/Medicines/Medicine (Medicinal) Products

Exemplary (consumable) pharmaceuticals (pharmaceutical products)/medicines (medicine [medicinal] products) are essentially any over-the-counter, or prescription only, type pharmaceutical (pharmaceutical product)/medicine (medicine [medicinal] product) which is consumed by a living entity for maintaining, or/and improving, or/and enhancing, or/and treating, health, well being, and quality of life thereof.

Any of the above described consumable substances (materials or objects) may be in essentially any physicochemical form of a solid, a semi-solid, a liquid, or a combination thereof.

### Real-Time Manner or Mode of Implementation

The term ‘real-time’, as used herein, refers to essentially any aspect of the disclosed method, or any aspect of the disclosed apparatus, which is (automatically or/and manually) performed, implemented, or used, at the same time, or at nearly the same time, with negligible or insignificant time lag, that a targeted (observed, tracked, monitored) event, scene, or situation of interest occurs or takes place. For example, the term ‘real-time’, as used herein, refers to essentially any action, activity, step, procedure, process, operation, function, or piece of equipment, of the disclosed method or apparatus, which is (automatically or/and manually) performed, implemented, or used, at the same time, or at nearly the same time, with negligible or insignificant time lag, that a targeted (monitored, tracked, observed) event, scene, or situation of interest occurs or takes place.

Steps or procedures, sub-steps or sub-procedures, and, equipment and materials, system units, system sub-units, devices, assemblies, sub-assemblies, mechanisms, structures, components, elements, and configurations, and, peripheral equipment, utilities, accessories, and materials, as well as operation and implementation, of exemplary embodiments, alternative embodiments, specific configurations, and, additional and optional aspects, characteristics, or features, thereof, of some embodiments of the present invention, are better understood with reference to the following illustrative description and accompanying drawings. Throughout the following illustrative description and accompanying drawings, same reference notation and terminology (i.e., numbers, letters, symbols, terms, and phrases) are consistently used and refer to same steps or procedures, sub-steps or sub-procedures, system units, system sub-units, devices, assemblies, sub-assemblies, mechanisms, structures, components, elements, and configurations, and, peripheral equipment, utilities, accessories, materials, components, elements, or/and parameters.

An aspect of some embodiments of the present invention is a method for characterizing life quality of a living entity via hyper-spectral imaging and analysis.

Referring now to the drawings, FIG. 1 is a flow diagram of an exemplary embodiment of the method (generally, indicated as, and referred to by, reference number 100), including the indicated exemplary steps (procedures) thereof, for characterizing life quality of a living entity via hyper-spectral imaging and analysis, and applications thereof. In FIG. 1, exemplary steps (procedures) of the exemplary embodiment shown are enclosed inside separate blocks (frames) which are assigned reference numbers, and are also indicated by dashed line arrows with accompanying reference numbers drawn between other exemplary steps. Accordingly, as shown, exemplary steps (procedures) are enclosed inside of blocks (frames) 112, 114, 116, 118, and 120, and indicated by dashed line curved arrows with accompanying reference numbers 122 and 124. As shown in FIG. 1, the exemplary embodiment of the method 100 includes the following exemplary steps (procedures).

In exemplary step (procedure) referenced by 112, there is acquiring hyper-spectral imaging data and information of: at least one anatomical feature of the living entity, and at least one substance, material, or object consumable by the living entity.

In exemplary step (procedure) referenced by 114, there is generating and maintaining a living entity-specific database containing data and information about the living entity.

In exemplary step (procedure) referenced by 116, there is processing the acquired living entity anatomical feature hyper-spectral imaging data and information, the acquired living entity consumable object hyper-spectral imaging data and information, and the data and information about the living entity.

In exemplary step (procedure) referenced by 118, there is using the processed data and information to generate living entity life quality data and information characteristic of at least one aspect of life quality of the living entity.

In exemplary step (procedure) referenced by 120, the living entity life quality data and information characteristic of at least one aspect of life quality of the living entity (generated via exemplary step 118) are fed back to the preceding step (procedure) 116, for example, as a way of updating or/and modifying step (procedure) 116 of processing the data-information.

In exemplary step (procedure) referenced by 122, the processed data and information of the acquired living entity anatomical feature hyper-spectral imaging data and information, the acquired living entity consumable object hyper-spectral imaging data and information, and the data and information about the living entity, obtained via exemplary step 116, are fed back to the preceding step (procedure) 114, for example, as a way of updating or/and modifying step (procedure) 114 of generating and maintaining a living entity-specific database containing data and information about the living entity.

In exemplary embodiments of method 10, in exemplary step (procedure) referenced by 30, the living entity life quality data and information is used to assist in managing life quality of the living entity. This exemplary step (procedure) may be considered as part of an exemplary application of some embodiments of the method 100.

Another aspect of some embodiments of the present invention is an apparatus for characterizing life quality of a living entity via hyper-spectral imaging and analysis, and applications thereof.

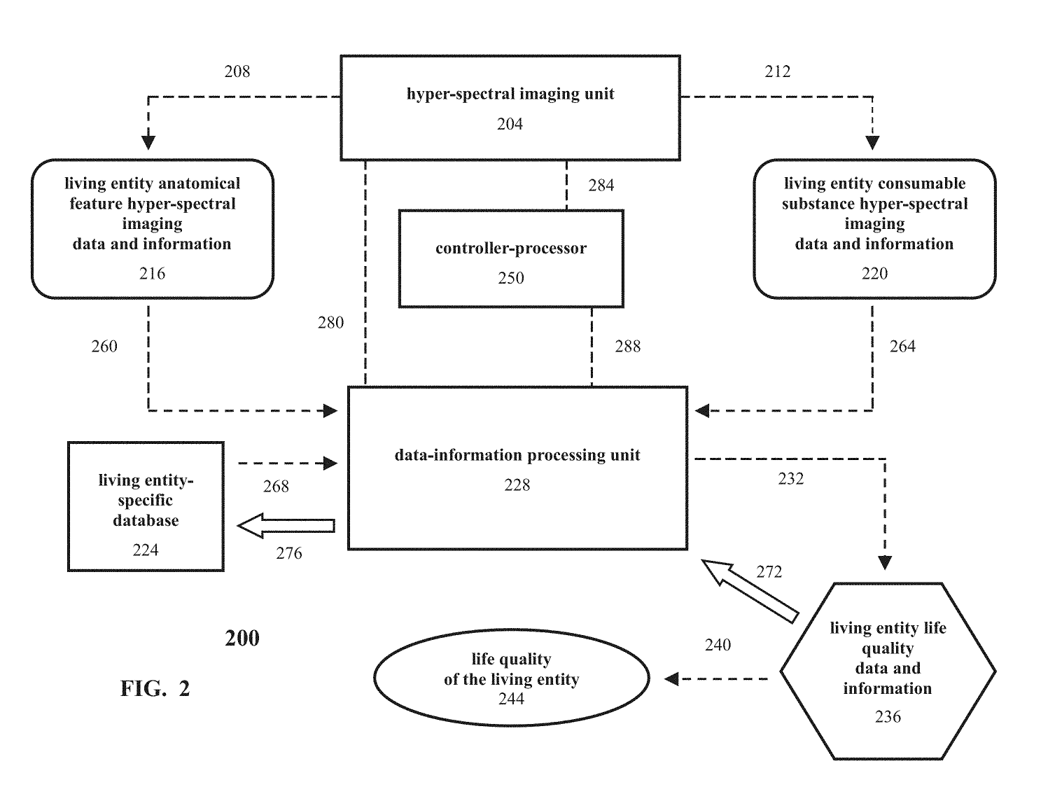

FIG. 2 is a (block-type) schematic diagram of an exemplary embodiment of an apparatus (generally, indicated as, and referred to by, reference number 200), including exemplary components and features thereof, for characterizing life quality of a living entity via hyper-spectral imaging and analysis, and applications thereof. The exemplary embodiment of the apparatus 200 is, in a non-limiting manner, particularly suitable for implementing the exemplary embodiment of the method 100 presented in FIG. 1. As shown in FIG. 2, the exemplary embodiment of the apparatus 200 includes the following exemplary components and functionalities thereof.

A hyper-spectral imaging unit 204, configured to acquire (indicated by dashed arrows 208 and 212) hyper-spectral imaging data and information of: at least one anatomical feature of the living entity 216, and at least one object consumable by the living entity 220.

A living entity-specific database 224 containing data and information about the living entity.

A data-information processing unit 228, configured to: process the acquired living entity anatomical feature hyper-spectral imaging data and information 216, the acquired living entity consumable object hyper-spectral imaging data and information 220, and the data and information about the living entity 224; and to generate (indicated by dashed arrow 232) living entity life quality data and information 236 characteristic of at least one aspect of life quality of the living entity.

In exemplary embodiments of apparatus 200, the data-information processing unit 228 is optionally configured to enable using (indicated by dashed arrow 240) the living entity life quality data and information 236 to assist in managing life quality of the living entity 244. This exemplary optional configuration may be considered as part of an exemplary application of apparatus 200.

In exemplary embodiments, apparatus 200 additionally includes a controller-processor 250 configured to control operation of the hyper-spectral imaging unit 204.

In exemplary embodiments, acquired living entity anatomical feature hyper-spectral imaging data and information 216 is transferred (indicated by dashed arrow 260) from hyper-spectral imaging unit 204 to data-information processing unit 228, and acquired living entity consumable object hyper-spectral imaging data and information 220 is transferred (indicated by dashed arrow 264) from hyper-spectral imaging unit 204 to data-information processing unit 228.

In exemplary embodiments, data and information about the living entity is transferred (indicated by dashed arrow 268) from living entity-specific database 224 to data-information processing unit 228.

In exemplary embodiments, living entity life quality data and information 236 is fed back (indicated by hollow arrow 272) to data-information processing unit 228.

In exemplary embodiments, data-information processing unit 228 feeds back (indicated by hollow arrow 276) processed data and information to living entity-specific database 224.

In exemplary embodiments, data and information, or/and control signals, are exchanged (indicated by dashed line 280) between data-information processing unit 228 and hyper-spectral imaging unit 204.

In exemplary embodiments, data and information, or/and control signals, are exchanged (indicated by dashed line 284) between controller-processor 250 and hyper-spectral imaging unit 204.

In exemplary embodiments, data and information, or/and control signals, are exchanged (indicated by dashed line 288) between data-information processing unit 228 and controller-processor 250.

### Temporal Aspects, Characteristics, and Features

Exemplary embodiments of the present invention are applicable for being implemented according to different ‘temporal’ manners or modes of operation, in the context of ‘time’. For example, as a function of time, for example, wherein the invention is implemented a number of times, for examples, according to a chronological sequence or plurality of a number of times, spanning a pre-determined period or length of time, wherein an exemplary pre-determined period or length of time is on the order of minutes, hours, days, weeks, months, or years.

Accordingly, in exemplary embodiments, exemplary method 100, and exemplary steps (procedures) thereof, for characterizing life quality of a living entity via hyper-spectral imaging and analysis, are performed as a function of time. For example, wherein exemplary method 10, and exemplary steps (procedures) thereof, are performed a number of times, for example, according to a chronological sequence or plurality of a number of times, spanning a pre-determined period or length of time.

Accordingly, in exemplary embodiments, exemplary apparatus 200, and exemplary components thereof, for characterizing life quality of a living entity via hyper-spectral imaging and analysis, are configured to be operative as a function of time. For example, wherein exemplary apparatus 200, and exemplary components thereof, are configured to be operative a number of times, for example, according to a chronological sequence or plurality of a number of times, spanning a pre-determined period or interval of time.

In exemplary embodiments, an exemplary pre-determined period or length of time is on the order of minutes, hours, days, weeks, months, or years.

In exemplary embodiments, exemplary method 100, or/and exemplary apparatus 200, is/are implemented in a manner corresponding to chronologically and periodically or sequentially characterizing life quality of the living entity via hyper-spectral imaging and analysis. Such a mode of implementation corresponds to chronologically and periodically or sequentially observing, tracking, or monitoring, the living entity life quality data and information 236 characteristic of at least one aspect of life quality of the living entity.

In exemplary embodiments, exemplary method 100, or/and exemplary apparatus 200, is/are implemented according to a real-time manner or mode of operation. For example, wherein exemplary method 10, and exemplary steps (procedures) thereof, are performed according to a real-time manner or mode of operation. For example, wherein exemplary apparatus 200, and exemplary components thereof, are configured to be operative according to a real-time manner or mode. For example, wherein essentially any action, activity, step, procedure, process, operation, function, or piece of equipment, of the exemplary method 100 or exemplary apparatus 200, may be (automatically or/and manually) performed, implemented, or used, at the same time, or at nearly the same time, with negligible or insignificant time lag, that a targeted (monitored, tracked, observed) event, scene, or situation of interest occurs or takes place, in the context of characterizing life quality of a living entity via hyper-spectral imaging and analysis.

### Spatial Aspects, Characteristics, and Features

Exemplary embodiments of the present invention are applicable for being implemented according to different ‘spatial’ manners or modes of operation, in the context of ‘equipment or hardware’ configurations and operations thereof. For example, in the form of a single, stand-alone multi-assembly or multi-device type of system, or, alternatively, in the form of (integrated) microelectromechanical (MEM) [chip level] components as part of any one of various different types or kinds of a smart or intelligent device, such as a smart or intelligent phone, a smart or intelligent wallet, a smart or intelligent watch, a smart or intelligent hand bracelet, or a smart or intelligent finger ring, among other possible smart or intelligent devices, used by people.

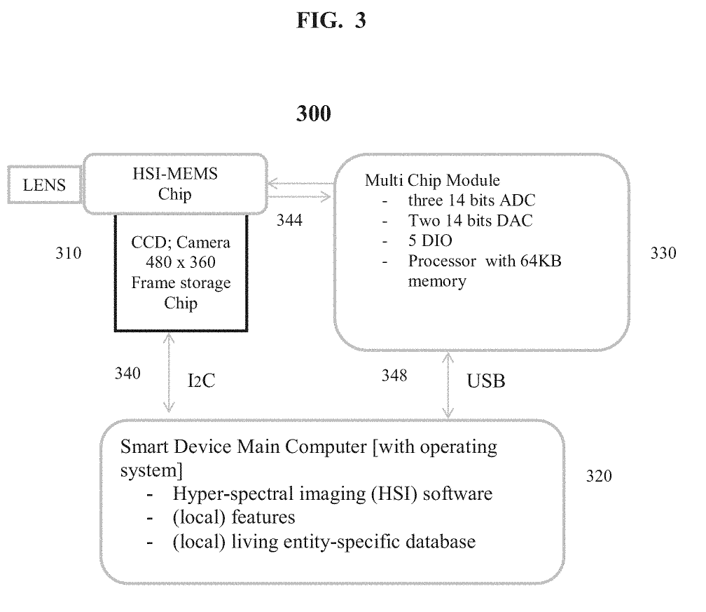

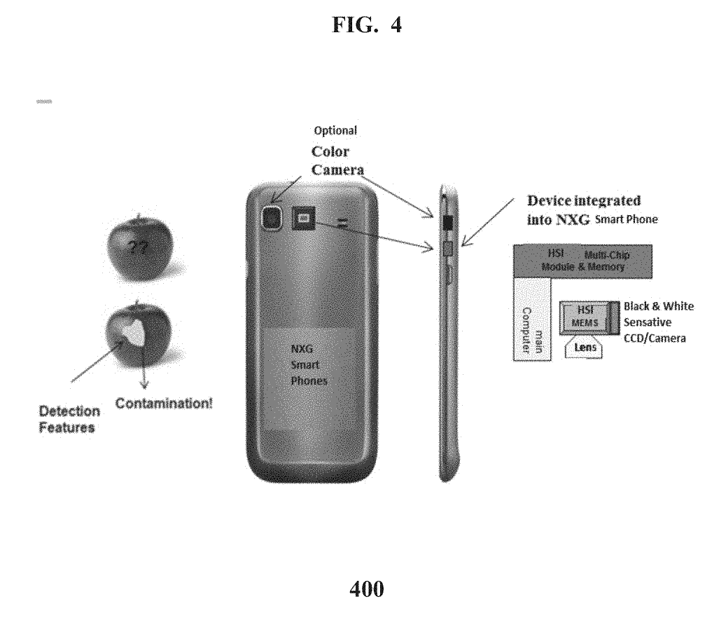

Accordingly, in exemplary embodiments, exemplary method 100, and exemplary steps (procedures) thereof, for characterizing life quality of a living entity via hyper-spectral imaging and analysis, are performed in the form of a single, stand-alone multi-assembly or multi-device type of system, or, alternatively, in the form of (integrated) microelectromechanical (MEM) [chip level] components as part of any one of various different types or kinds of a smart or intelligent device (for example, as shown in FIG. 3), such as a smart or intelligent phone (for example, as shown in FIG. 4), a smart or intelligent wallet, a smart or intelligent watch, a smart or intelligent hand bracelet, or a smart or intelligent finger ring, among other possible smart or intelligent devices.

Accordingly, in exemplary embodiments, exemplary apparatus 200, and exemplary components thereof, for characterizing life quality of a living entity via hyper-spectral imaging and analysis, are configured to be operative in the form of a single, stand-alone multi-assembly or multi-device type of system, or, alternatively, in the form of (integrated) microelectromechanical (MEM) [chip level] components as part of any one of various different types or kinds of a smart or intelligent device (for example, as shown in FIG. 3), such as a smart or intelligent phone (for example, as shown in FIG. 4), a smart or intelligent wallet, a smart or intelligent watch, a smart or intelligent hand bracelet, or a smart or intelligent finger ring, among other possible smart or intelligent devices.

Additional Structural or/and Functional (Operational) Aspects, Characteristics, and Features

Exemplary embodiments of the present invention, for example, as illustratively described herein above in the context of exemplary method 100 (FIG. 1) and exemplary apparatus 200 (FIG. 2), for characterizing life quality of a living entity via hyper-spectral imaging and analysis, include the following exemplary additional structural or/and functional (operational) aspects, characteristics, and features.

In exemplary embodiments, for example, via exemplary step (procedure) 112, there is acquiring hyper-spectral imaging data and information of at least one anatomical feature of the living entity. In exemplary embodiments, the living entity may be any living (live) entity, such as a living (live) human being, a living (live) animal, or, a living (live) plant, for example, as listed and described hereinabove.

In exemplary embodiments, the at least one anatomical feature of the living entity may be essentially any anatomical feature, or part of the body, of the living entity which is or can be (partly or fully) exposed and at least partly viewable by an observer, either with or without movement or manipulation of the body of the living entity or/and removal of something covering the body of the living entity, for example, as listed and described hereinabove.

In exemplary embodiments, there is acquiring hyper-spectral imaging data and information of at least one substance consumable by the living entity. In exemplary embodiments, the at least one substance consumable by the living entity may be essentially any substance, material, or object, which is consumable or usable (i.e., may be consumed or used) by a living entity in a manner where the consumable substance, material, or object, contacts (touches, comes into direct contact with, or is ingested by) at least part of the body of the living entity, for example, as listed and described hereinabove.

In exemplary embodiments, for example, via exemplary step (procedure) 114, there is generating and maintaining a living entity-specific database containing data and information about the living entity. In exemplary embodiments, the living entity-specific database contains data and information, including hyper-spectral imaging data and information, about the hereinabove listed and described anatomical features of the living entity, and, data and information, including hyper-spectral data and information, about the hereinabove listed and described substances consumable by the living entity. Such a living entity-specific database serves as a ‘reference’ type of database containing reference data and information, including reference hyper-spectral data and information, about the living entity.

In exemplary embodiments, for example, via exemplary step (procedure) 116, there is processing the acquired living entity anatomical feature hyper-spectral imaging data and information, the acquired living entity consumable object hyper-spectral imaging data and information, and the data and information about the living entity.

In exemplary embodiments, such processing includes performing at least some of the following exemplary steps (procedures), via the data-information processing unit.

Forming sets and databases of interferogram images from the acquired living entity anatomical feature hyper-spectral imaging data and information, the acquired living entity consumable object hyper-spectral imaging data and information, and the data and information about the living entity.

Segmenting (via analyzing and classifying), for example, as a function of time: (i) elements of the acquired living entity anatomical feature hyper-spectral imaging data and information, (ii) elements of the acquired living entity consumable object hyper-spectral imaging data and information, and (iii) elements of the living entity-specific database containing the (reference) data and information about the living entity.

Such segmenting, for example, as a function of time, is done in order to generate or form: (i) segmented (analyzed and classified) acquired living entity anatomical feature hyper-spectral imaging data and information, (ii) segmented (analyzed and classified) acquired living entity consumable object hyper-spectral imaging data and information, and (iii) segmented (analyzed and classified) reference data and information about the living entity, as a function of time.

In exemplary embodiments, following the preceding segmenting, for example, via exemplary step (procedure) 118, there is using the processed data and information to generate living entity life quality data and information characteristic of at least one aspect of life quality of the living entity.

This step includes, for example, correlating (via using software programs based on, for example, fuzzy logic decision making, and matching/mismatching, algorithms) at least two of: (i) the (time dependent) segmented (analyzed and classified) acquired living entity anatomical feature hyper-spectral imaging data and information, (ii) the (time dependent) segmented (analyzed and classified) acquired living entity consumable object hyper-spectral imaging data and information, and (iii) the (time dependent) segmented (analyzed and classified) reference data and information about the living entity.

Such correlating, for example, as a function of time, is done in order to generate the living entity life quality data and information characteristic of at least one aspect of life quality of the living entity, for example, as a function of time.

In exemplary embodiments, for example, following the preceding correlating, for example, via exemplary step (procedure) 120, at least some elements of the generated living entity life quality data and information characteristic of at least one aspect of life quality of the living entity are fed back to the preceding step (procedure) 116, for example, as a way of updating or/and modifying step (procedure) 116 of processing the acquired living entity anatomical feature hyper-spectral imaging data and information, the acquired living entity consumable object hyper-spectral imaging data and information, and the data and information about the living entity.

In exemplary embodiments, for example, also following the preceding correlating, for example, via exemplary step (procedure) 122, at least some elements of the processed data and information of the acquired living entity anatomical feature hyper-spectral imaging data and information, the acquired living entity consumable object hyper-spectral imaging data and information, and the data and information about the living entity, obtained via exemplary step 116, are fed back to the preceding step (procedure) 114, for example, as a way of updating or/and modifying step (procedure) 114 of generating and maintaining the living entity-specific database containing data and information about the living entity.

In exemplary embodiments, for example, via exemplary step (procedure) 130, the living entity life quality data and information is used to assist in managing life quality of the living entity. This exemplary step (procedure) may be considered as part of an exemplary application of some embodiments of the present invention.

### Exemplary Applications

As described hereinabove, exemplary embodiments of the present invention are applicable for being implemented according to different ‘spatial’ manners or modes of operation, in the context of ‘equipment or hardware’ configurations and operations thereof. For example, in the form of a single, stand-alone multi-assembly or multi-device type of system, or, alternatively, in the form of (integrated) microelectromechanical (MEM) [chip level] components as part of any one of various different types or kinds of a smart or intelligent device, such as a smart or intelligent phone, a smart or intelligent watch, a smart or intelligent wallet, or, a smart or intelligent ring, among other possible smart or intelligent devices, used by people.

Accordingly, in exemplary embodiments, exemplary method 100, and exemplary steps (procedures) thereof, for characterizing life quality of a living entity via hyper-spectral imaging and analysis, are performed in the form of a single, stand-alone multi-assembly or multi-device type of system, or, alternatively, in the form of (integrated) microelectromechanical (MEM) [chip level] components as part of any one of various different types or kinds of a smart or intelligent device (for example, as shown in FIG. 3), such as a smart or intelligent phone (for example, as shown in FIG. 4), a smart or intelligent wallet, a smart or intelligent watch, a smart or intelligent hand bracelet, or a smart or intelligent finger ring, among other possible smart or intelligent devices.

Similarly, in exemplary embodiments, exemplary apparatus 200, and exemplary components thereof, for characterizing life quality of a living entity via hyper-spectral imaging and analysis, are configured to be operative in the form of a single, stand-alone multi-assembly or multi-device type of system, or, alternatively, in the form of (integrated) microelectromechanical (MEM) [chip level] components as part of any one of various different types or kinds of a smart or intelligent device (for example, as shown in FIG. 3), such as a smart or intelligent phone (for example, as shown in FIG. 4), a smart or intelligent wallet, a smart or intelligent watch, a smart or intelligent hand bracelet, or a smart or intelligent finger ring, among other possible smart or intelligent devices.

FIG. 3 is a (block-type) schematic diagram of an exemplary apparatus (generally, indicated as, and referred to by, reference number 300) for characterizing life quality of a living entity via hyper-spectral imaging and analysis, and applications thereof, highlighting an exemplary ‘chip level’ type design and construction, configurable and operable with integrated microelectromechanical (MEM) [chip level] components as part of an exemplary smart device.

As shown in FIG. 3, exemplary apparatus 300 includes the following exemplary components.

A hyper-spectral imaging unit 310, configured to acquire hyper-spectral imaging data and information of: at least one anatomical feature of a living entity, and at least one object consumable by the living entity. Hyper-spectral imaging unit 310 includes an HSI-MEMS chip, a CCD—camera chip, and a lens assembly.

A data-information processing unit 320, configured as a smart device main computer including: an operating system, hyper-spectral imaging (HSI) software, (local) features data and information, and a (local) living entity-specific database (for example, 224 of FIG. 2) containing data and information about the living entity.

Data-information processing unit 320 is configured to: process the acquired living entity anatomical feature hyper-spectral imaging data and information (for example, 216 of FIG. 2), the acquired living entity consumable object hyper-spectral imaging data and information (for example, 220 of FIG. 2), and the data and information about the living entity (for example, 224 of FIG. 2).

Data-information processing unit 320 is also configured to generate (for example, 232 of FIG. 2) living entity life quality data and information (for example, 236 of FIG. 2) characteristic of at least one aspect of life quality of the living entity.

In exemplary embodiments of apparatus 300, the data-information processing unit 320 is optionally configured to enable using (for example, 240 of FIG. 2) the living entity life quality data and information (for example, 236 of FIG. 2) to assist in managing life quality of the living entity (for example, 244 of FIG. 2). This exemplary optional configuration may be considered as part of an exemplary application of apparatus 300.

Exemplary apparatus 300 also includes a controller-processor 330 configured to control operation of the hyper-spectral imaging unit 310.

In exemplary embodiments, data and information, or/and control signals, are exchanged (indicated by the term “I2C” and the two-headed arrow 340) between data-information processing unit 320 and hyper-spectral imaging unit 310.

In exemplary embodiments, data and information, or/and control signals, are exchanged (indicated by the pair of arrows 344) between controller-processor 330 and hyper-spectral imaging unit 310.

In exemplary embodiments, data and information, or/and control signals, are exchanged (indicated by the term “USB” and the two-headed arrow 348) between data-information processing unit 320 and controller-processor 330.

FIG. 4 is a schematic diagram illustrating an exemplary apparatus (generally, indicated as, and referred to by, reference number 400) for characterizing life quality of a living entity via hyper-spectral imaging and analysis, and applications thereof, as an example of the apparatus 300 of FIG. 3, configured into an exemplary smart phone.

Each of the following terms written in singular grammatical form: ‘a’, ‘an’, and ‘the’, as used herein, means ‘at least one’, or ‘one or more’. Use of the phrase ‘one or more’ herein does not alter this intended meaning of ‘a’, ‘an’, or ‘the’. Accordingly, the terms ‘a’, ‘an’, and ‘the’, as used herein, may also refer to, and encompass, a plurality of the stated entity or object, unless otherwise specifically defined or stated herein, or, unless the context clearly dictates otherwise. For example, the phrases: ‘a unit’, ‘a device’, ‘an assembly’, ‘a mechanism’, ‘a component’, ‘an element’, and ‘a step or procedure’, as used herein, may also refer to, and encompass, a plurality of units, a plurality of devices, a plurality of assemblies, a plurality of mechanisms, a plurality of components, a plurality of elements, and, a plurality of steps or procedures, respectively.

Each of the following terms: ‘includes’, ‘including’, ‘has’, ‘having’, ‘comprises’, and ‘comprising’, and, their linguistic/grammatical variants, derivatives, or/and conjugates, as used herein, means ‘including, but not limited to’, and is to be taken as specifying the stated component(s), feature(s), characteristic(s), parameter(s), integer(s), or step(s), and does not preclude addition of one or more additional component(s), feature(s), characteristic(s), parameter(s), integer(s), step(s), or groups thereof. Each of these terms is considered equivalent in meaning to the phrase ‘consisting essentially of’.

Each of the phrases ‘consisting of’ and ‘consists of’, as used herein, means ‘including and limited to’.

The phrase ‘consisting essentially of’, as used herein, means that the stated entity or item (system, system unit, system sub-unit, device, assembly, sub-assembly, mechanism, structure, component, element, or, peripheral equipment, utility, accessory, or material, method or process, step or procedure, sub-step or sub-procedure), which is an entirety or part of an exemplary embodiment of the disclosed invention, or/and which is used for implementing an exemplary embodiment of the disclosed invention, may include at least one additional ‘feature or characteristic’ being a system unit, system sub-unit, device, assembly, sub-assembly, mechanism, structure, component, or element, or, peripheral equipment, utility, accessory, or material, step or procedure, sub-step or sub-procedure), but only if each such additional ‘feature or characteristic’ does not materially alter the basic novel and inventive characteristics or special technical features, of the claimed entity or item.

The term ‘method’, as used herein, refers to steps, procedures, manners, means, or/and techniques, for accomplishing a given task including, but not limited to, those steps, procedures, manners, means, or/and techniques, either known to, or readily developed from known steps, procedures, manners, means, or/and techniques, by practitioners in the relevant field(s) of the disclosed invention.

Throughout this disclosure, a numerical value of a parameter, feature, characteristic, object, or dimension, may be stated or described in terms of a numerical range format. Such a numerical range format, as used herein, illustrates implementation of some exemplary embodiments of the invention, and does not inflexibly limit the scope of the exemplary embodiments of the invention. Accordingly, a stated or described numerical range also refers to, and encompasses, all possible sub-ranges and individual numerical values (where a numerical value may be expressed as a whole, integral, or fractional number) within that stated or described numerical range. For example, a stated or described numerical range ‘from 1 to 6’ also refers to, and encompasses, all possible sub-ranges, such as ‘from 1 to 3’, ‘from 1 to 4’, ‘from 1 to 5’, ‘from 2 to 4’, ‘from 2 to 6’, ‘from 3 to 6’, etc., and individual numerical values, such as ‘1’, ‘1.3’, ‘2’, ‘2.8’, ‘3’, ‘3.5’, ‘4’, ‘4.6’, ‘5’, ‘5.2’, and ‘6’, within the stated or described numerical range of ‘from 1 to 6’. This applies regardless of the numerical breadth, extent, or size, of the stated or described numerical range.

Moreover, for stating or describing a numerical range, the phrase ‘in a range of between about a first numerical value and about a second numerical value’, is considered equivalent to, and meaning the same as, the phrase ‘in a range of from about a first numerical value to about a second numerical value’, and, thus, the two equivalently meaning phrases may be used interchangeably. For example, for stating or describing the numerical range of room temperature, the phrase ‘room temperature refers to a temperature in a range of between about 20° C. and about 25° C.’, and is considered equivalent to, and meaning the same as, the phrase ‘room temperature refers to a temperature in a range of from about 20° C. to about 25° C.’.

The term ‘about’, as used herein, refers to ±10% of the stated numerical value.

The phrase ‘operatively connected’, as used herein, equivalently refers to the corresponding synonymous phrases ‘operatively joined’, and ‘operatively attached’, where the operative connection, operative joint, or operative attachment, is according to a physical, or/and electrical, or/and electronic, or/and mechanical, or/and electro-mechanical, manner or nature, involving various types and kinds of hardware or/and software equipment and components.

It is to be fully understood that certain aspects, characteristics, and features, of the present invention, which are illustratively described and presented in the context or format of a plurality of separate embodiments, may also be illustratively described and presented in any suitable combination or sub-combination in the context or format of a single embodiment. Conversely, various aspects, characteristics, and features, of the present invention, which are illustratively described and presented in combination or sub-combination in the context or format of a single embodiment, may also be illustratively described and presented in the context or format of a plurality of separate embodiments.

Although the present invention has been illustratively described and presented by way of specific exemplary embodiments thereof, and examples thereof, it is evident that many alternatives, modifications, and variations, thereof, will be apparent to those skilled in the art. Accordingly, it is intended that all such alternatives, modifications, and variations, fall within, and are encompassed by, the scope of the appended claims.

All patents, patent applications, and publications, cited or referred to in this specification are herein incorporated in their entirety by reference into the specification, to the same extent as if each individual patent, patent application, or publication, was specifically and individually indicated to be incorporated herein by reference. In addition, citation or identification of any reference in this specification shall not be construed or understood as an admission that such reference represents or corresponds to prior art of the present invention. To the extent that section headings are used, they should not be construed as necessarily limiting.

### REFERENCES

1. WIPO PCT Patent Application International Publication No. WO 2012/150557, published Nov. 8, 2012, of PCT Patent Application No. PCT/IB2012/052195, filed May 2, 2012, of same applicant/assignee as the present disclosure, entitled: “Microelectromechanical System (MEMS) And (MEM) Optical Interferometer For Hyper-Spectral Imaging And Analysis”.
2. WIPO PCT Patent Application International Publication No. WO 2008/099407, published Aug. 21, 2008, of PCT Patent Application No. PCT/IL2008/000205, filed Feb. 14, 2008, of same applicant/assignee as the present invention, entitled: “Hyper-spectral Imaging And Analysis Of A Sample Of Matter, And Preparing A Solution Or Suspension Therefrom”.
3. WIPO PCT Patent Application International Publication No. WO 2007/0990540, published Sep. 7, 2007, of PCT Patent Application. No. PCT/IL2007/000268, filed Mar. 1, 2007, of same applicant/assignee as the present invention, entitled: “Processing And Analyzing Hyper-spectral Image Data And Information Via Dynamic Database Updating”.
4. U.S. Pat. No. 7,411,682, to Moshe, of same applicant/assignee as the present invention, entitled: “Real Time High Speed High Resolution Hyper-Spectral Imaging”.
5. U.S. Pat. No. 6,697,510, to Moshe, of same applicant/assignee as the present invention, entitled: “Method For Generating Intra-particle Crystallographic Parameter Maps And Histograms Of A Chemically Pure Crystalline Particulate Substance”.
6. U.S. Pat. No. 6,694,048, to Moshe, of same applicant/assignee as the present invention, entitled: “Method For Generating Intra-particle Morphological Concentration/Density Maps And Histograms Of A Chemically Pure Particulate Substance”.
7. U.S. Pat. No. 6,438,261, to Moshe, et al., of same applicant/assignee as the present invention, entitled: “Method Of In-situ Focus-Fusion Multi-layer Spectral Imaging And Analysis”.
8. U.S. Pat. No. 6,091,843, to Horesh, et al., of same applicant/assignee as the present invention, entitled: “Method Of Calibration And Real-time Analysis Of Particulates”.
9. U.S. Pat. No. 5,880,830, to Schechter, of same applicant/assignee as the present invention, entitled: “Spectral Imaging Method For On-line Analysis Of Polycyclic Aromatic Hydrocarbons In Aerosols”.

1. A method for characterizing changes in a life quality of a living entity over a predetermined time period via hyper-spectral imaging and analysis, the method comprising:
automatically transmitting control signals to a hyper-spectral imaging unit for acquiring, over the predetermined time period:
hyper-spectral imaging data and information of at least one anatomical feature of the living entity, at preselected intervals spanning the predetermined time period, and
hyper-spectral imaging data and information of at least one substance consumable by the living entity, at said preselected intervals spanning the predetermined time period, wherein said information of at least one substance consumable by the living entity relates to said at least one consumable substance prior to said substance coming into contact with the living entity;
storing said hyper-spectral imaging data and information in a memory;
generating and maintaining a living entity-specific database containing data and information about the living entity at said preselected intervals spanning the predetermined time period;
storing said living entity-specific database in a memory;
using a hyper-spectral image processor for:
segmenting at least a portion of said hyper-spectral imaging data and said data of said living entity-specific database, to provide segmented data;
applying fuzzy logic to find correlations in said segmented data and to identify changes in at least one aspect of life quality of the living entity over the predetermined time period; and
generating an output containing information assisting in managing life quality of the living entity.

2. The method of claim 1, wherein said acquiring, said storing hyper-spectral imaging data and information, said generating and maintaining a living entity-specific database, said storing said living entity-specific database, said segmenting, said applying fuzzy logic, said generating an output, and said using, are performed according to a chronological sequence or plurality of a number of times, spanning the pre-determined period of time.

3. The method of claim 2, wherein said pre-determined period of time is on order of minutes, hours, days, weeks, months, or years.

4. The method of claim 1, wherein said acquiring, said storing hyper-spectral imaging data and information, said generating and maintaining a living entity-specific database, said storing said living entity-specific database, said segmenting, said applying fuzzy logic, said generating an output, and said using, are performed according to a real-time manner or mode of operation.

5. The method of claim 1, wherein said using a hyper-spectral image processor further includes forming sets and databases of interferogram images from said acquired living entity anatomical feature hyper-spectral imaging data and information, said acquired living entity consumable object hyper-spectral imaging data and information, and said data and information about the living entity.

6. The method of claim 1, wherein said segmenting includes analyzing and classifying, as a function of time: (i) elements of said acquired living entity anatomical feature hyper-spectral imaging data and information, (ii) elements of said acquired living entity consumable object hyper-spectral imaging data and information, and (iii) elements of said living entity-specific database containing reference data and information about the living entity.

7. The method of claim 6, wherein said applying said fuzzy logic includes correlating at least two of: (i) said segmented acquired living entity anatomical feature hyper-spectral imaging data and information, (ii) said segmented acquired living entity consumable object hyper-spectral imaging data and information, and (iii) said segmented reference data and information about the living entity.

8. The method of claim 7, wherein following said correlating, at least some elements of said generated living entity life quality data and information characteristic of at least one aspect of life quality of the living entity are fed back to said processor, so as to update or/and modify processing of said acquired living entity anatomical feature hyper-spectral imaging data and information, said acquired living entity consumable object hyper-spectral imaging data and information, and said data and information about the living entity.

9. The method of claim 7, wherein following said correlating, at least some elements of said processed data and information of said acquired living entity anatomical feature hyper-spectral imaging data and information, said acquired living entity consumable object hyper-spectral imaging data and information, and said data and information about the living entity, are fed back to said generating and maintaining, so as to update or/and modify said generating and maintaining said living entity-specific database containing data and information about the living entity.

10. The method of claim 1, wherein said changes in living entity life quality data and information characteristic of at least one aspect of life quality of the living entity generated and maintained over the predetermined time period is used for assisting in managing life quality of the living entity.

11. An apparatus for characterizing changes in life quality of a living entity over a predetermined time period via hyper-spectral imaging and analysis, the apparatus comprising:
a hyper-spectral imaging unit, having a circuit configured to acquire, at preselected intervals spanning the predetermined time period, hyper-spectral imaging data and information of: at least one anatomical feature of the living entity and at least one substance consumable by the living entity, wherein said information of at least one substance consumable by the living entity relates to said at least one consumable substance prior to said substance coming into contact with the living entity;
a memory storing a living entity-specific database containing data and information about the living entity at said preselected intervals spanning the predetermined time period; and
a hyper-spectral image processor, having a circuit configured to:
segment at least a portion of said hyper-spectral imaging data and said data of said living entity-specific database, to provide segmented data;
apply fuzzy logic to find correlations in said segmented data and to identify changes in at least one aspect of life quality of the living entity over the predetermined time period; and
generate an output containing information assisting in managing life quality of the living entity.

12. The apparatus of claim 11, wherein said hyper-spectral imaging unit, and said hyper-spectral image processor are configured to operate according to a chronological sequence or plurality of a number of times, spanning the pre-determined period of time.

13. The apparatus of claim 11, wherein said hyper-spectral image processor is additionally configured to process said data and information by forming sets and databases of interferogram images from said acquired living entity anatomical feature hyper-spectral imaging data and information, said acquired living entity consumable object hyper-spectral imaging data and information, and said data and information about the living entity.

14. The apparatus of claim 11, wherein said hyper-spectral image processor is configured to process said data and information by segmenting, as a function of time: (i) elements of said acquired living entity anatomical feature hyper-spectral imaging data and information, (ii) elements of said acquired living entity consumable object hyper-spectral imaging data and information, and (iii) elements of said living entity-specific database containing reference data and information about the living entity.

15. The apparatus of claim 14, wherein said hyper-spectral imaging processor is further configured to apply fuzzy logic to at least two of: (i) said segmented acquired living entity anatomical feature hyper-spectral imaging data and information, (ii) said segmented acquired living entity consumable object hyper-spectral imaging data and information, and (iii) said segmented reference data and information about the living entity.

16. The apparatus of claim 11, wherein said hyper-spectral imaging processor is configured to update or/and modify at least some elements of said processed data and information of said acquired living entity anatomical feature hyper-spectral imaging data and information, said acquired living entity consumable object hyper-spectral imaging data and information, and said data and information about the living entity.

17. The apparatus of claim 11, further comprising a controller-processor configured to control said hyper-spectral imaging unit.

18. The apparatus of claim 11, wherein said living entity life quality data and information is fed back to said hyper-spectral imaging processor.

19. The apparatus of claim 11, wherein said hyper-spectral imaging processor is configured to feed back said processed data and information to said living entity-specific database.

20. The apparatus of claim 11, wherein said hyper-spectral imaging processor is configured to enable using said living entity life quality data and information to assist in managing life quality of the living entity.

21. The method of claim 1, wherein said preselected intervals are on an order selected from minutes, hours, days, weeks, months, and years.

22. The method of claim 1, wherein said acquiring, said storing said hyper-spectral imaging data and information, said generating and maintaining a living entity-specific database, said storing said living entity-specific database, and said using a hyper-spectral image processor are performed on a smart device.

23. The method of claim 22, wherein said smart device is one of a smart phone, a smart wallet, a smart watch, a smart bracelet, and a smart ring.
